(19) United States
(12) Patent Application Publication
(10) Pub. No.: US 2016/0095742 A1
(43) Pub. Date: Apr. 7, 2016

(54) Facial Bands

(71) Applicant: Adriana Knies, Plantation, FL (US)

(72) Inventor: Adriana Knies, Plantation, FL (US)

(21) Appl. No.: 14/506,558

(22) Filed: Oct. 3, 2014

(51) Int. Cl. A61F 7/02

(52) U.S. Cl. CPC A61F 7/02; A61F 2007/0003

(57) ABSTRACT

Facial Bands is an apparatus designed to relieve pressure and pain in the human face. This apparatus is made of two flexible bands, which includes various pouches with a material that can be heated or cooled to relieve pressure and pain on the human face; such as but not limited to the lower lip area, chin, cheeks, jawbone line, the sides of the face, and the temples. The two parts interlock and create one complete apparatus, with one band starting at the lower part of the chin and jawbone going upwards and latching at the top of the head, the other lower band piece goes from the lower lip around the jawline, going around the neck and latching at the back of the head.

### BACKGROUND OF INVENTION

| Document Identifier | Publication Date |
|---|---|
| 1,964,655 | Jun. 26, 1934 |
| 3,491,761 | Jan. 27, 1970 |
| 4,044,773 | Aug. 30, 1977 |
| 4,190,054 | Feb. 26, 1980 |
| 4,326,533 | Apr. 27, 1982 |
| 4,347,848 | Sep. 7, 1982 |
| 4,523,353 | Jun. 18, 1985 |
| 4,527,566 | Jul. 9, 1985 |
| 4,688,572 | Aug. 25, 1987 |
| 4,805,620 | Feb. 21, 1989 |
| 4,835,950 | Jun. 6, 1989 |
| 5,514,170 | May 7, 1996 |
| 5,603,729 | Feb. 18, 1997 |
| 5,641,325 | Jun. 24, 1997 |
| 5,723,063 | Mar. 3, 1998 |
| 5,887,437 | Mar. 30, 1999 |
| 6,036,004 | Mar. 14, 2000 |
| 6,051,159 | Apr. 18, 2000 |
| 6,126,683 | Oct. 3, 2000 |
| 6,261,314 | Jul. 17, 2001 |
| 7,083,839 | Aug. 1, 2006 |

### RELATED U.S. PATENT DOCUMENTS

| | | | |
|---|---|---|---|
| Application Number | Filing Date | Patent Number | Issue Date |

| | |
|---|---|
| 14/299,376(From the same | Jun. 9, 2014 |
| applicant) | |

### BRIEF SUMMARY OF THE INVENTION

The present invention is a pain and pressure relief device for the human face. This apparatus is made of two flexible bands, which includes various pouches with material that can be heated or cooled, utilized to relieve pressure and pain on the human face, such as but not limited to lip area, chin, cheeks, jawbone line, side of the face and the temple. The two parts interlock and create one complete apparatus, with one band starting at the lower part of the chin and jawbone going upwards, and latching at the top of the head, the other lower band piece goes from the lower lip around the head within the alignment of the jawbone around the neck and latching at the back of the head. The 2 bands interlock where they intersect. With this application of the apparatus the user will get relief from pain and pressure but not limited to facial injuries, facial pain, tooth aches, migraine; the use of the apparatus is described here on these documents as Drawings and Brief Description of the Several Views of the Drawings, furthermore its material also provides packs that can be heated or cooled. The method is comprised of; (FIG. 1)—this part is applied to the surface of the skin adjacent to the skin, from underneath the chin, going up on both right and left sides of the face, and around the jawbone and going upwards to the top of the head where the right and the left of the bands join and latch together, this part contains a total of 7 pouches with material that can be heated or cooled, that each will hold 1 pouch with rectangular edges, on the right and on left side of the band, the pouches with material that can be heated or cooled start just above the jawbone, one pocket just underneath the chin; (FIG. 2)—this part is applied to the surface of the skin adjacent to the skin, from above the chin, around the jawbone and going back on both sides the right and left around the neck and towards the back of the head where it latches, this part contains a total of 5 pouches with material that can be heated or cooled, that each will hold 1 pouch with material that can be heated or cooled with rectangular edges, on the right and on left side of the band, just above the jawbone, and one pouch just underneath the lower lip; (FIG. 3)—represents the pouches with material that can be heated or cooled with rectangular edges.

### BRIEF DESCRIPTION OF THE SEVERAL VIEWS OF THE DRAWINGS

FIG. 1—is a perspective view of the one of the parts of the apparatus, this part is applied to the surface of the skin adjacent to the skin, from underneath the chin, going up on both right and left sides of the face, and around the jawbone going upwards to the top of the head where they latch. This part contains a flexible band, which includes various pouches with material that can be heated or cooled, to relieve pressure and pain on the human face, such as but not limited to lip area, chin, cheeks, jawbone line, side of the face and the temple. There are 3 pouches in each side, each holds the material that can be heated or cooled on the right and on left side of the band, the pouches start just above the jawbone, and one pouch just underneath the chin; its parts (a) showing the latching system, (b) pouches with material that can be heated or cooled; (c) the material that it is flexible and it expands and contracts giving the user an increase of light resistance and comfort (I.S.) interlocking system showing on FIG. 1, where FIG. 2 will joined creating one apparatus showing on FIG. 4.

FIG. 2—this part is applied to the surface of the skin adjacent to the skin, from beneath the lip area, around the jawbone and going back on both the right and left sides toward the back of the head where it latches, this part contains 2 pouches in each side, each will hold a material that can be heated or cooled and one pouch in the middle making the fifth pouch just underneath the lower lip; its part (a) showing the latching system, (b) pouches with material that can be heated or cooled; (c) the material that it is flexible and it expands and contracts giving the user an increase of light resistance and comfort

FIG. 3—represents pouches with material that can be heated or cooled

FIG. 4—is a perspective view of the apparatus interlocked showing its users physical location in forward position:

FIG. 5 is a perspective view of the apparatus interlocked and showing its users physical location in side position

FIG. 6 is a perspective view of the apparatus interlocked and showing its user physical location in a rear position.

### BRIEF DETAILED DESCRIPTION OF THE INVENTION

As shown in FIG. 1,

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

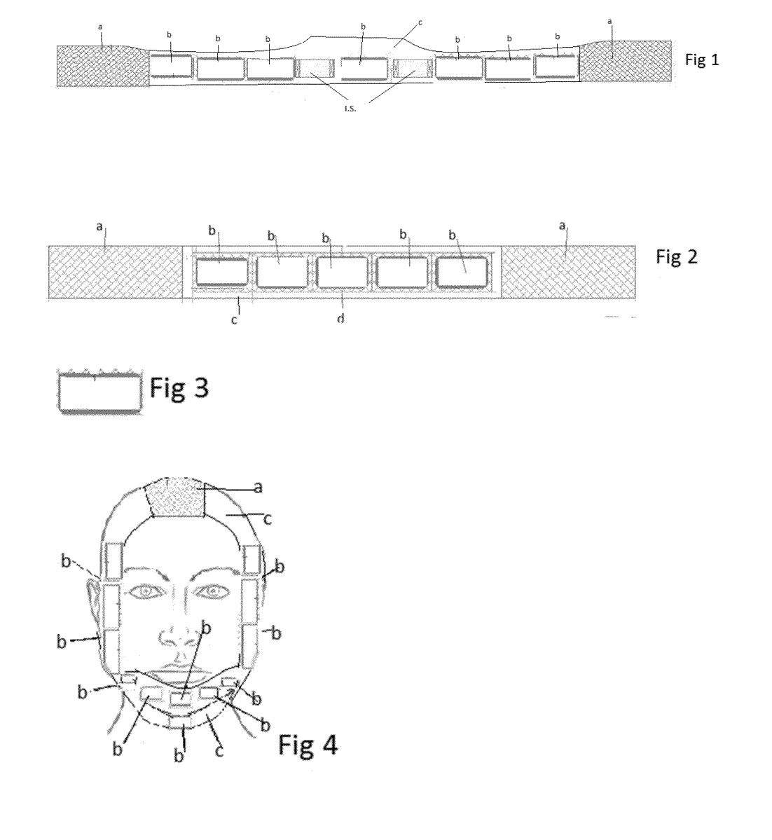

As shown in FIG. 2,

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

As shown in FIG. 3,

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

As shown in FIG. 4,

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

As shown in FIG. 5,

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

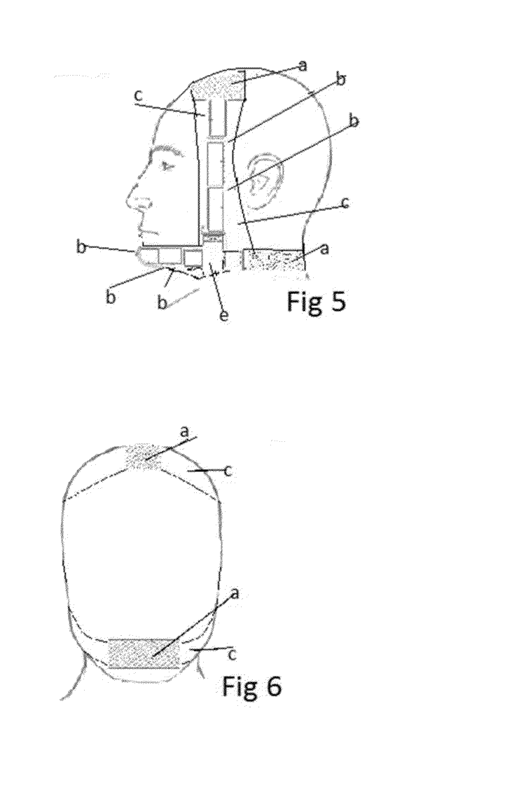

As shown in FIG. 6,

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A method of relieving pressure and pain in the human face, comprised of the following steps: The method comprised of; (FIG. 1)—this part is applied to the surface of the skin adjacent to the skin, from underneath the chin, going up on both right and left sides of the face, and around the jawbone going upwards to the top of the head where the right and the left sides of the band join and latch together. This part contains a total of 7 pouches with pouches with material that can be heated or cooled, that each will hold one pouch with rectangular edges, on the right and on left side of the band, the pouches with pouches with material that can be heated or cooled start just above the jawbone, and one pouch just underneath the chin; (FIG. 2)—This part is applied to the surface of the skin adjacent to the skin, from above the chin, around the jawbone and going back both right and left sides around the neck to the back of the head and where it latches, this part contains 2 pouches in each side, that each will hold pouches with material that can be heated or cooled, and stays on the right and on left side of the band, just above the jawbone, with one more pouch just underneath the lower lip; (FIG. 3)—pouches with material that can be heated or cooled.
